(19) United States
(12) Patent Application Publication
(10) Pub. No.: US 2008/0269641 A1
(43) Pub. Date: Oct. 30, 2008

(54) METHOD OF USING A GUIDEWIRE WITH STIFFENED DISTAL SECTION

(75) Inventors: Donagh O'Shaughnessy, Kinvara (IE); Noel Coyle, Craughwell (IE)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/739,834

(22) Filed: Apr. 25, 2007

(51) Int. Cl. A61M 25/01

(52) U.S. Cl. 600/585

(57) ABSTRACT

A method of treating a vascular condition includes inserting at least one locally stiffened guidewire into a vessel, advancing a distal section of the guidewire to a position adjacent a treatment site and providing a stiffening portion of the distal section to a location adjacent the treatment site. The method further includes disposing a delivery catheter over the inserted guidewire, advancing a distal portion of the delivery catheter to the treatment site, the distal portion carrying at least one stent and deploying the stent at the treatment site.

### FIELD OF THE INVENTION

This invention relates generally to medical guidewires, and particularly to the design and methods of using guidewires having a distal section with increased stiffening that corresponds to an intravascular treatment site.

### BACKGROUND

Cardiovascular disease, including atherosclerosis, is a leading cause of death in the U.S. A number of methods and devices for treating coronary heart disease have been developed, including a broad array of catheters and guidewires and minimally invasive methods for using them. Catheter-based delivery systems are routinely used to introduce stents and other medical devices into the cardiovascular system for both therapeutic and diagnostic purposes. Many of the catheters used in such delivery systems, including over-the-wire catheters and rapid exchange catheters, require a small diameter steerable guidewire to direct the catheter through the vascular system.

Typically, a short guidewire is inserted into the vascular system through a needle puncture in an artery, such as the femoral, brachial, or radial artery. Then, a longer guidewire is threaded through the vascular system until the distal end of the guidewire is adjacent to the treatment site. The distal portion of the catheter is slipped over the proximal end of the guidewire, and the catheter is advanced along the guidewire through the vasculature until the distal portion of the catheter is positioned at the target site. The position of the catheter end may be determined by common visualization methods such as fluoroscopy or ultrasound.

Steerable guidewires may also be used as a core member to construct low profile intravascular devices such as so-called “balloon-on-a-wire” or fixed-wire dilatation catheters, filter guidewires and occluder guidewires. Medical procedures using catheters that require guidewires include percutaneous transluminal angioplasty, stent delivery, atherectomy, treatment of aneurysms and other catheterization procedures.

In order to perform well, a guidewire must have sufficient columnar strength and rigidity so that it can be pushed through the vasculature of the patient without bending back on itself (also known as prolapse) or kinking. Kinking of the guidewire often is accompanied by a permanent deformation of the guidewire where the kink occurred. Different steerable guidewires are provided with a variety of stiffnesses in the distal region to accommodate different catheterization procedures and different patient vascular anatomies. For example, guidewires with relatively stiff distal regions are typically used to guide a stent delivery catheter. However, if a guidewire distal region is too stiff; it may not be able to negotiate highly tortuous vasculature. In order to balance the need for both flexibility and columnar strength, guidewires are frequently constructed to have a relatively rigid proximal section and a more flexible distal section, as shown in U.S. Pat. No. 4,545,390 to Leary.

One drawback to the more flexible distal sections of a guidewire is that the advancing distal section may not move in the desired direction when steered. Flexible distal ends also have a greater tendency to prolapse. These problems are especially troublesome when the clinician is advancing a guidewire to delivery and implant a stent or other device at a bifurcated treatment site.

It would be desirable, therefore, to deploy a stent over a steerable guidewire that has a variation in bending stiffness along a distal section that overcomes the problems of guidewires found often to be either too stiff or too flexible for stent delivery. Such a guidewire can provide safe, responsive navigation through blood vessels and side branches, and offers a stiffer distal portion to prevent the guidewire prolapsing and to improve delivery of stents, especially the orientation of bifurcated stents in a bifurcation stenosis.

### SUMMARY OF THE INVENTION

One embodiment of the invention provides a method of treating a vascular condition that includes inserting at least one locally stiffened guidewire into a vessel. A flexible distal portion provides safe advancement of a distal section of the guidewire to a position adjacent a treatment site. The method further includes disposing a delivery catheter over the inserted guidewire, advancing a distal portion of the delivery catheter to the treatment site, the distal portion carrying at least one stent and deploying the stent at the treatment site. The stiffening portion of the distal section prevents the guidewire prolapsing during navigation and during the delivery of a stent. The stiffening portion of the distal section guides the relatively stiff stent into the treatment site, without guidewire prolapse.

### BRIEF DESCRIPTION OF THE DRAWINGS

The present invention is illustrated by the accompanying drawings of various embodiments and the detailed description given below. The drawings should not be taken to limit the invention to the specific embodiments but are for explanation and clarity. The detailed description and drawings are merely illustrative of the invention rather than limiting, the scope of the invention being defined by the appended claims and equivalents thereof. The foregoing aspects and other attendant advantages of the present invention will become more readily appreciated by the detailed description taken in conjunction with the accompanying drawings, which are not to scale.

FIG. 1 illustrates a lateral, partially schematic view of one embodiment of a locally stiffened guidewire in accordance with the present invention;

FIG. 2 illustrates a lateral, partially schematic view of another embodiment of a locally stiffened guidewire in accordance with the present invention;

FIGS. 3 to 6 illustrate the use of one embodiment of a locally stiffened guidewire during the delivery and implantation of a bifurcated stent; and

FIG. 7 is a flow chart of one embodiment of a method of using a locally stiffened guidewire in accordance with the present invention.

### DETAILED DESCRIPTION

Throughout this specification, like reference numbers refer to like structures. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

Generally, steerable guidewires of the present invention are of a length and dimension typical of conventional guidewires. The guidewires of the present invention have a proximal stiff section and a softer more flexible distal section. Typically, the guidewires will have a length in the range of about 170 to 300 cm and a diameter in the range of about 0.30 mm (0.012 inch) to about 0.89 mm (0.035 inch). In one example, a typical diameter for guidewires used in catheterization and treatment of coronary arteries is 0.36 mm (0.014 inch). In order to provide a more flexible distal section, the guidewires of the present invention may have a distal body or core section that is tapered in the distal direction. As the diameter decreases along the length of the guidewire distal section, lateral flexibility increases. The term “taper” herein refers to any reduction in diameter over a length. Such a reduction in diameter may have a constant rate or angle, or it may comprise a series of steps or regions having different cylindrical portions and/or different taper angles.

The variable diameters in the distal section of the guidewire may be produced by a plunge-type centerless grinding procedure as is known in the art. The guidewires of the present invention provide proximal sections that are relatively more stiff than the distal sections. Conversely, the distal sections are more flexible than the proximal sections in order for the distal tip to traverse through the vasculature. Generally, the stiffness of the guidewire will decrease from the proximal end to the distal end and the flexibility of the guidewire will increase from the proximal end to the distal end. The stiffness and the flexibility of the guidewire are determined by such factors as the diameter of the guidewire and the materials composing the guidewire.

The guidewires of the present invention may include an outer covering or coating composed of an elastomeric material. The material for the outer covering or coating may be, for example, polyethylene, polyvinyl chloride, polyester, polypropylene, polyamide, polyurethane, polystyrene, fluoropolymer resin, silicone rubber, and combinations, thereof. The guidewires of the present invention may also include a lubricious coating to aid in traversing the vasculature.

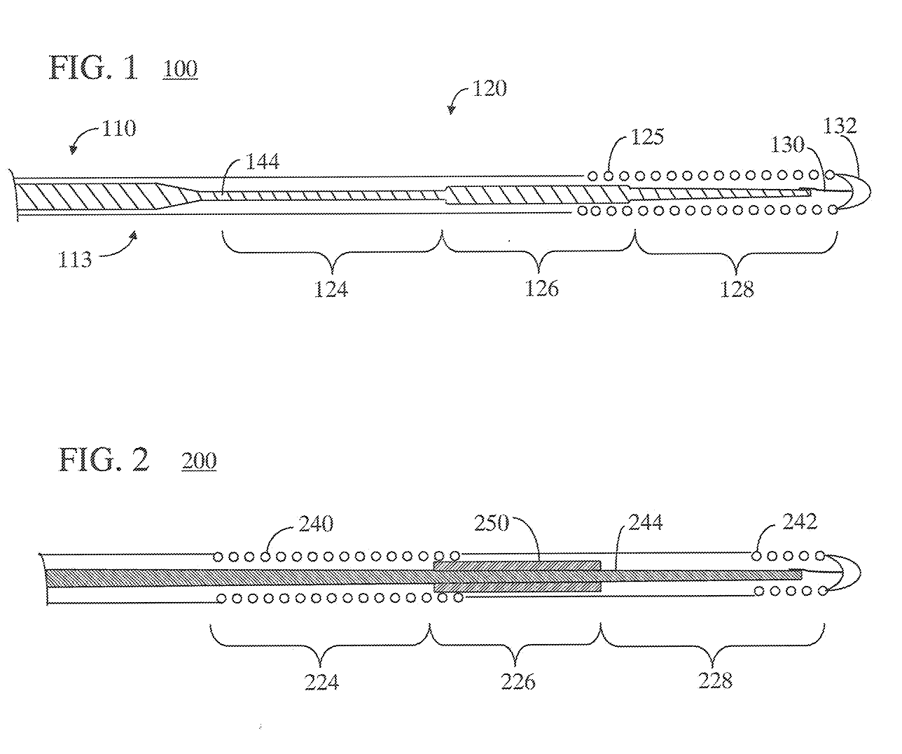

As described above, the guidewires of the present invention have a proximal section and a softer, more flexible distal section. However, as described in more detail below and illustrated in FIGS. 1-2, the softer more flexible distal section of the guidewires of the present invention includes a stiffening element for locally stiffening a portion of the distal section of the guidewire.

FIG. 1 illustrates one embodiment of a locally stiffened guidewire 100 in accordance with the present invention. Guidewire 100 includes a proximal section 110 and a distal section 120. In one embodiment, proximal section 110 tapers toward distal section 120. Proximal section 110 has a relatively high columnar strength sufficient to transmit to distal section 120 substantially all of the axial and rotational forces applied to the portion of proximal section 110 extending outside of the patient. The length of proximal section 110 is selected according to the intended use of guidewire 100. In one embodiment of the invention, the diameter of proximal section 110 may be up to about 0.36 mm (0.014 inch). However, the diameter of proximal section 110 will be as small as required to fit slidably within suitable catheters or other medical devices.

Proximal section 110 may be composed of any conventional biocompatible guidewire material. Proximal section 110 may comprise one or more metals selected from stainless steel, titanium, cobalt-chromium based alloy, nickel titanium (nitinol), tungsten, tungsten alloy, tantalum or other bio-compatible rigid or semi-rigid material.

Distal section 120 commonly includes a core wire 144 that is a reduced-diameter portion of the wire comprising proximal section 110. Alternatively, distal section 120 may be a separate element attached to distal end 113 of proximal section 110. The diameter of distal section 120 is typically substantially equal to the diameter of proximal section 110. A tubular element 125 such as a coil spring surrounds core wire 144.

Distal section 120 includes a proximal portion 124, a distal portion 128 and a stiffening portion 126. Distal portion 128 is attached to an end cap 132 at the distal end of tubular element 125 by safety/shaping ribbon 130, as will be understood by one of skill in the art of guidewires. End cap 132 may comprise adhesive, solder or welded metal. At least a portion of tubular element 125 is typically composed of a radiopaque material.

Stiffening portion 126 is disposed between proximal portion 124 and distal portion 128. In one embodiment, portions 124, 126 and 128 of distal section 120 are defined by the relative stiffness of each respective portion. In an embodiment, proximal portion 124 has a first stiffness greater than a second stiffness of distal portion 128. In this same embodiment, stiffening portion 126 has a third stiffness greater than the first stiffness of proximal portion 124.

In one embodiment, the stiffness of portions 124, 126 and 128 is determined by varying the diameter of core wire 144. In this embodiment, stiffening portion 126 has a diameter that is greater than the diameter of proximal section 124. In one exemplary embodiment, proximal portion 124 has a diameter of 0.19 mm (0.0075 inch) and stiffening portion 126 has a diameter of 0.23 mm (0.009 inch). In such an embodiment, distal portion 128 has a diameter smaller than the diameter of proximal portion 124. In another embodiment, stiffening portion 126 has a diameter that is at least 10 percent greater than the diameter of proximal portion 124. In another embodiment, the diameter of stiffening portion 126 is greater than the diameter of both proximal portion 124 and distal portion 128.

Distal section 120 has a length that is approximately 5 to 50 percent of the total length of guidewire 100. The lengths of portions 124, 126 and 128 may be determined by the specific application of the particular guidewire. Considerations for determining the length of distal section 120 as well as portions 124, 126 and 128 include, but are not limited to, the distance from the entry point to the treatment site, the location of the treatment site, and the tortuousness of the vasculature leading to and adjacent the treatment site. The length of stiffening portion 126 may be determined based on the treatment site. Generally, the length of stiffening portion 126 is chosen to span the entire treatment side. In one embodiment, stiffening portion 126 has a length equal to or greater than the distance between the proximal and distal ends of the treatment site.

FIG. 2 illustrates locally stiffened guidewire 200, another embodiment in accordance with the present invention. Guidewire 200 is similar to guidewire 100 illustrated in FIG. 1. However, in this embodiment, proximal portion 224 of distal section 220 has a first stiffness that is the same as or similar to the second stiffness of distal portion 228. In this embodiment, stiffening portion 226 has a third stiffness that is greater than the stiffness for both proximal portion 224 and distal portion 228. In this embodiment, proximal portion 224 and distal portion 228 are substantially more flexible than stiffening portion 226. In one embodiment, proximal portion 224 and distal portion 228 include flexible coils 240, 242, respectively, and stiffening portion 226 is composed of an elongated substantially linear core wire 244 having a stiffening sleeve 250. Stiffening sleeve 250 is coaxially disposed about core wire 244. Stiffening sleeve 250 may comprise a metallic or polymeric based material or combinations thereof. Suitable metallic materials may be, for example, stainless steel, titanium, cobalt-chromium based alloy, nickel titanium (nitinol), tungsten, tungsten alloy, tantalum or other bio-compatible rigid or semi-rigid metallic based materials. Suitable polymeric materials may be, for example, polyester, polytetrafluoroethylene (PTFE), polyethylene, polyvinyl chloride, polypropylene, polyamide, polyurethane, polystyrene and other elastomers.

In another embodiment, the difference in stiffness between stiffening portion 226 and proximal and distal portions 224, 228 is determined by the physical properties of the respective materials. In an example, the material of proximal portion 224 and distal portion 228 is relatively more flexible than the material of stiffening portion 226. Such variation in physical properties may be provided by using different materials in different portions. In an embodiment, stiffening portion 226 may comprise tungsten or a tungsten alloy, while proximal and distal portions 224 and 228 may comprise TiNi (nitinol) or stainless steel. The different material portions may be joined together by sleeves, e.g. sleeve 250 wherein the sleeve is a thin, flexible material that contributes little to the bending stiffness of the core wire assembly.

In another embodiment, the desirable variation in physical properties between stiffening portion 226 and proximal and distal portions 224, 228 may be provided by variations in heat-treatment or work-hardening of a single core wire material. For example, proximal and distal portions 224, 228 may be annealed to enhance bending flexibility, and stiffening portion 226 may be cold-worked or otherwise treated to harden the material and thus increase bending stiffness. Such treatments may be applied to any metals such as those mentioned above with respect to proximal section 110.

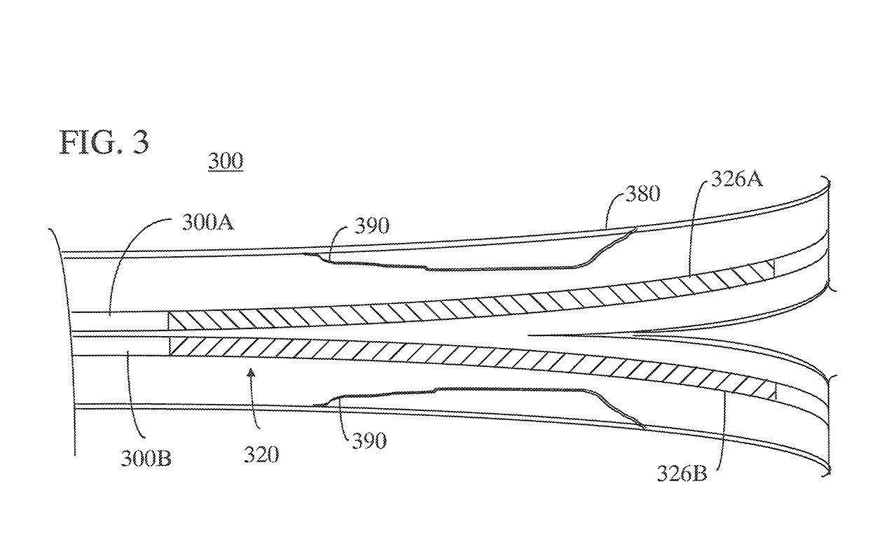

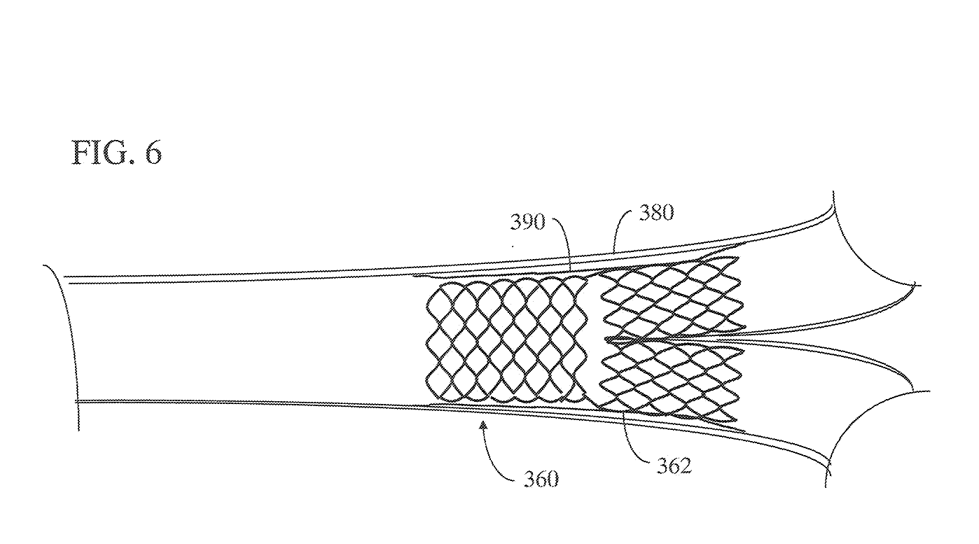

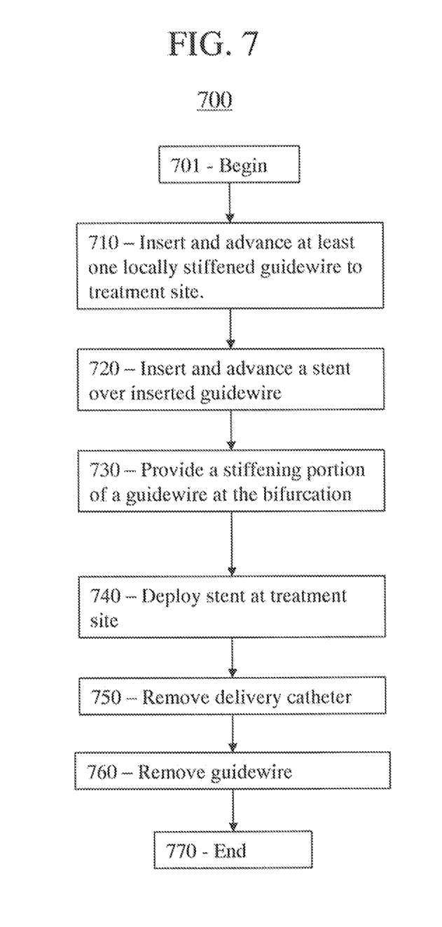

FIGS. 3 to 6 illustrate the use of one embodiment of a locally stiffened guidewire during the delivery and implantation of a bifurcated stent, in accordance with one embodiment of the invention. Reference will be made to FIG. 7 which illustrates a flow chart of one embodiment of a method of treating a vascular condition with a locally stiffened guidewire system, at 700. Method 700 is provided in relation to using locally stiffened guidewires 300 to treat a bifurcated lesion 390. Those with skill in the art will appreciate that the locally stiffened guidewire may be used in treating locations within the patient other than a bifurcated location. Method 700 begins at 701.

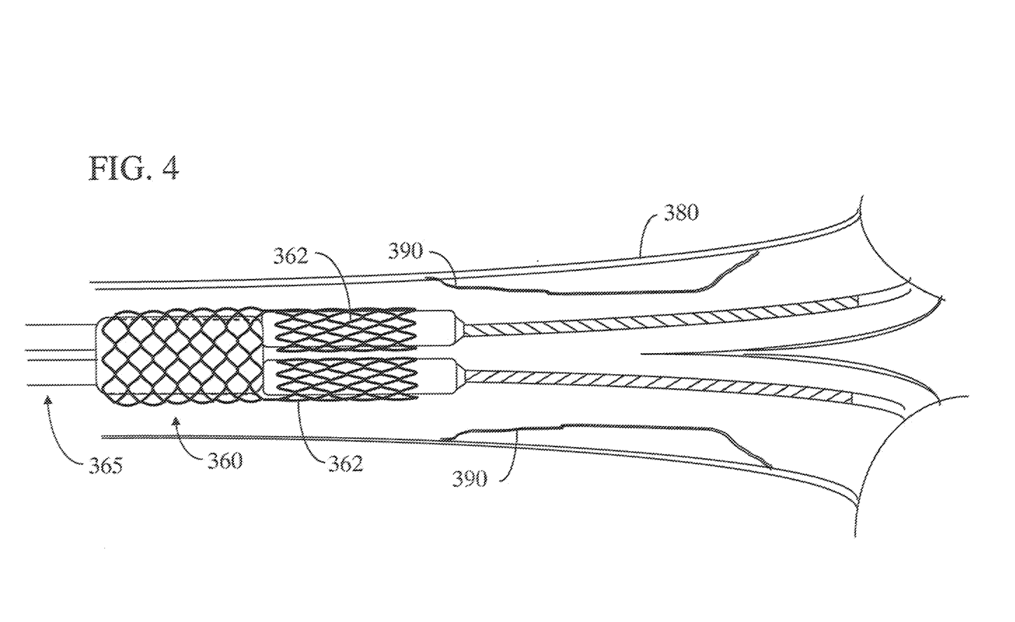

Locally stiffened guidewires 300A, 300B are inserted into the vasculature and advanced to a bifurcated treatment site 380 having a lesion 390 near the bifurcation. Locally stiffened guidewires 300A, 300B are the same as or similar to the guidewires described above and illustrated in FIGS. 1-2. Guidewires 300A, 300B are advanced through the vasculature until the stiffening portions 326A, 326B of guidewires 300A, 300B are proximate lesion 390. The advancement of guidewires 300A, 300B may be observed using standard fluoroscopy techniques. In one embodiment, stiffening portions 326A, 326B include radiopaque indicia to aid in visualization while aligning stiffening portions 326A, 326B within lesion 390. The radiopaque indicia may include, e.g., disposing radiopaque material along the entire length of stiffening portions 326A, 326B. Alternately, discrete radiopaque marker bands may be disposed at distal and proximal ends of stiffening portions 326A, 326B. Stiffening portions 326A, 326B of guidewires 300A, 300B will aid in the delivery and implantation of bifurcated stent 360, as illustrated in FIGS. 4 to 6.

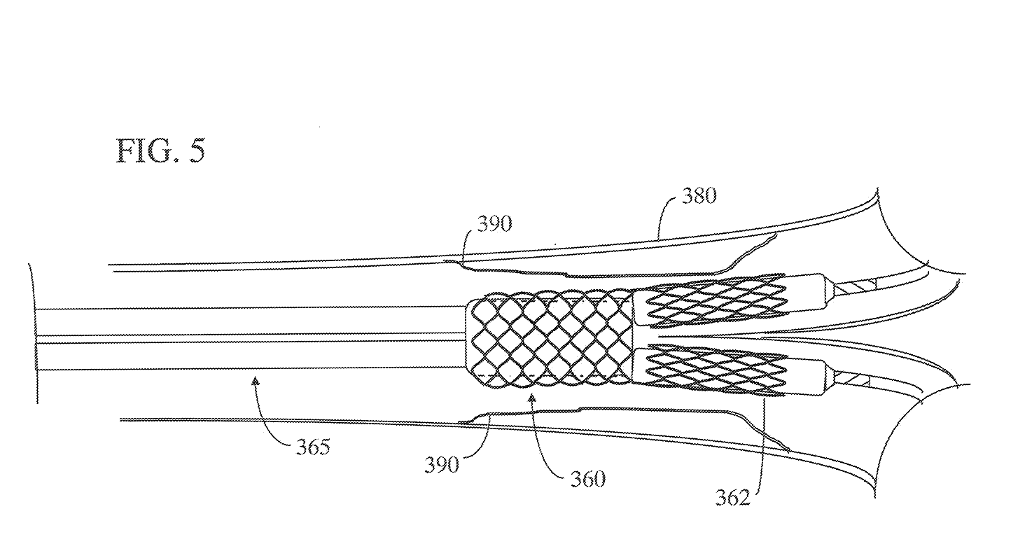

Next, a bifurcated stent delivery catheter 365 carrying a bifurcated stent 360 is disposed on guidewires 300A, 300B and inserted into the vasculature (Block 720). The inserted delivery catheter 360 is advanced over guidewires 300A, 300B until the bifurcated stent 360 is proximate the lesion 390 located at the bifurcation. As shown in FIG. 4, the inserted guidewires 300A, 300B act as tracks or rails to guide the delivery catheter to the treatment site. Next, localized stiffening portions 326 of guidewires 300A, 300B provide a stiffened section of the guidewires for the orientation and placement of stent legs 362 within the bifurcation at the treatment site, without guidewire prolapse occurring as best seen in FIG. 5 (Block 730).

Bifurcated stent 360 is deployed at the treatment site into contact with the vessel wall (Block 740). In one embodiment, stent 360 is deployed by inflating balloons of delivery catheter 365. In other embodiments, a self expanding stent may be used whereby the stent is deployed by retracting a sheath (not shown) as is known in the art. Next, the balloon is deflated and the delivery catheter is removed from the vasculature (Block 750). Guidewires 300A, 300B are removed from the vasculature (Block 760) leaving the implanted bifurcated stent 360 behind at the bifurcated treatment site. Method 700 ends at 770.

While the invention has been described with reference to particular embodiments, it will be understood by one skilled in the art that variations and modifications may be made in form and detail without departing from the spirit and scope of the invention.

What is claimed is:

1. A method of treating a vascular condition, the method comprising:
inserting into a vessel at least one locally stiffened guidewire having a locally stiffened portion;
advancing the locally stiffened portion of the at least one locally stiffened guidewire to a location adjacent a treatment site;
disposing a delivery catheter over the inserted at least one locally stiffened guidewire;
advancing the delivery catheter until a distal catheter portion carrying at least one stent is disposed about the locally stiffened portion of the at least one locally stiffened guidewire; and
deploying the stent at the treatment site.

2. The method of claim 1 wherein a first locally stiffened guidewire is inserted and advanced until a first locally stiffened portion is disposed adjacent a first branch of a bifurcated vessel treatment site and a second locally stiffened guidewire is inserted and advanced until a second locally stiffened portion is disposed adjacent a second branch of a bifurcated vessel treatment site.

3. The method of claim 2 wherein advancing the delivery catheter comprises advancing a delivery catheter having a bifurcated stent.

4. The method of claim 3 wherein the bifurcated stent comprises a body portion, a first leg portion and a second leg portion.

5. The method of claim 3 wherein advancing the delivery catheter further comprises advancing the first leg portion of the bifurcated stent over the first locally stiffened portion and advancing the second leg portion of the bifurcated stent over the second locally stiffened portion.

6. The method of claim 5 wherein deploying the stent at the treatment site comprises deploying a first leg portion of the bifurcated stent into contact with a first bifurcated portion of the vessel wall and deploying a second leg portion of the bifurcated stent into contact with a second bifurcated portion of the vessel wall.

7. The method of claim 6 wherein deploying the bifurcated stent comprises inflating a balloon portion of the delivery catheter.

8. The method of claim 1 wherein the locally stiffened portion comprises radiopaque indicia.

9. The method of claim 1 further comprising retracting the delivery catheter in response to the deploying of the stent.

10. The method of claim 9 further comprising retracting the at least one guidewire in response to retracting the delivery catheter.
